(19) United States
(12) Patent Application Publication
(10) Pub. No.: US 2011/0020252 A1
(43) Pub. Date: Jan. 27, 2011

(54) METHOD OF LONG LASTING HUMAN SKIN TANNING

(76) Inventors: Totada R. Shantha, McDonough, GA (US); Lauren Shantha, McDonough, GA (US); Usha T. Shantha Martin, Macon, GA (US)

Correspondence Address: PATWRITE LLC, 408 W. MAIN ST., MARSHALLTOWN, IA, 50158-5759, US

(21) Appl. No.: 12/896,033

(22) Filed: Oct. 1, 2010

(51) Int. Cl. A61K 8/49; A61Q 17/04

(52) U.S. Cl. 424/59

(57) ABSTRACT

The invention relates to the use of Rifampin and its related compound as a method of producing long-lasting, effective skin tanning for human and animal. The invention relates to methods of accelerating and facilitating the tanning of the skin using Rifampin along with use of the other tanning facilitators and accelerants with or without UVR.

### FIELD OF THE INVENTION

The invention relates to the method for long lasting human skin tanning using. It is particularly related to compositions which enhance the tanning of the skin. The invention relates to a method of accelerating tanning of the skin using compositions.

### BACKGROUND OF THE INVENTION

Tanning is a cosmetic and aesthetic method used in the western culture, especially by Caucasian females, to enhance the beauty and psychological uplifting feeling of being beautiful. In the United States, indoor tanning, tanning at the beaches and swimming pools is a thriving industry. There are an estimated 18,000 tanning salons in the US, excluding, tanning beds in health spas and personal tanning devices in homes. Nearly 70 percent of tanning salons patrons are Caucasian females, aged between 16 to 29 years. Nearly 28 million people tan indoors in the United States annually. Of these, 2.3 million are teens. The indoor tanning industry has estimated revenue of $5 billion, a fivefold increase since 1992. Millions of people go to beaches for their vacation to get a tan by exposing themselves to natures' ultraviolet radiation (UVR) emitted by the sun.

In humans, an increase of skin pigmentation of the epidermis is called tanning. This is physiologically stimulated by ultraviolet rays (UVR). UVR-induced skin darkening involves an increase in the number of melanocytes when there is stimulation of melanin synthesis and melanocytes dendricity, a crucial morphological feature required for melanin transfer to keratinocytes which imparts color that we see as a tan. It is the Ultra Violet rays (UVR) that play a role in tanning by the sun or in tanning booths.

The ozone layer of the Earth filters out most of the suns' harmful rays, but the ultraviolet rays (type UVA and UVB) do penetrate the ozone layer. These rays tan and damage the skin. Blocking these rays prevent sunburn, photoaging, wrinkles, increases lowered immunity against infection, and prevents skin cancers. UVA rays make up 95% of UV radiation with wavelength 320-400 nanometers (nm). They can penetrate into the deepest layer of the skin. UVB rays have a shorter wavelength of 290-320 nm and can penetrate the epidermis which damages DNA, cause sunburns, swelling, and most skin cancers. Skin cancers begin, when UV radiation damages, the DNA that controls skin cell growth. Both forms of UV rays plays a role in skin cancer which the UVB rays are a more potent trigger of skin cancers. One or two blistering sunburns early in life can greatly increase the lifetime risk of developing melanoma (Titus-Ernstoff L. Perry A E. Spencer S K. Gibson J. Ding J. Cole B, Ernstoff M S. Multiple primary melanoma: two-year results from a population-based study. Arch Dermotol. 2006 April; 142 (4):433-8).

Indoor ultraviolet (UV) tanners are 74 percent more likely to develop melanoma than those who have never tanned indoors. Additionally, the more time a person spends tanning indoors, higher the risk for development of skin cancer. Melanoma is the deadliest form of skin cancer. It killed approximately 8,650 Americans in 2009. UVR causes the highest number of skin precancerous and cancerous lesions in the body. Ultraviolet radiation from tanning machines, lamps, and tanning beds emit the most dangerous forms of cancer-causing radiation according to a 2009 report, which was released by the International Agency for Research on Cancer (IARC, affiliated with the World Health Organization). The IARC includes solar radiation in its list of the most dangerous types of cancer-causing substances including others.

Today the cosmetic method of tanning contributes to the ultraviolet light (UVL) burden of the epidermal skin layers which may lead to skin cancer. The preferred method is “Sunless” tanning methods that avoid UVL exposure. Sunless tanning may represent a safe alternative to UVR which it does not produce desired long lasting effects. The cancer causing effects of UVR related tanning, repeated exposure to the sun and tanning booth-lamps are discouraged or minimized. The present invention provides long lasting durable desired tan, lowering the number of exposures to UVR light in tanning booths, thus, reduces the incidence of UVR related cancers of the skin.

Attempts are made to develop UVR sunless tanning methods. The following are some of the methods used.

Dihydroxyacetone: A popular sunless tanning products contain the active ingredient Dihydroxyacetone (DHA). It is a 3-carbon sugar that unites and coalesces covalently with basic groups of proteins in the stratum corneum of the epidermis. This results in a browning reaction known as the Maillard reaction. The phenomenon of skin coloration with DHA was discovered in the mid-1950s at the University of Cincinnati by Eva Wittgenstein. She noted, when the patients took the DHA orally, it left pigmented spots when it came in contact on the skin. (Wittgenstein E, Berry H K. Staining of skin with dihydroxyacetone. Science 1960; 132: 894-895. Goldman L, Barkoff j, Blaney D, et al. Investigative studies with the skin coloring agents dihydroxyacetone and glyoxal. J Invest Dermatol 1960; 35:161-164). The tanning and cosmetic industry took notice. The first sunless tanning product was brought to the market in 1959 which does not use UVR Melanocytes to make the skin a tan color. In recent years the American public has become more aware of sunless tanning methods which are an alternative to UVL tanning. The newer sunless tanning formulations provide a cosmetically improved color where their use has been augmented.

It was found that the majority of individuals undergoing spray-on sunless tanning do not alter their sun exposure or their sunscreen use as a result of using sunless tanning. However, the greater parts of individuals who have used UVL tanning beds report a decrease in their tanning bed usage as the result of sunless tanning. Hence, the physicians should advocate the use of sunless tanning to their patients which means the decreasing UVL that exposure has dangerous effects on the skin.

The degree of tanning of human epidermis by the sun rays or UVR emitting devices depends upon the complexion of the individual. UVR tanning of the skin is the due the formations of melanin in melanocytes, a skin pigment. Its transfer from the melanocyte projections to the keratinocytes on surface of the skin, and its oxidation darkening as described in U.S. Pat. No. 3,988,437. The level to which an individual is tanned upon exposure to sunlight or artificial sources of ultra violet light depends upon the duration, intensity, and exposure to sunlight or such sources.

The ultraviolet energy absorbed by the skin produces a redness reaction (erythema) of the skin. Therefore, an attempt at getting a deeper and more intense tan by prolonged exposure to sunlight or other artificial sun sources can cause excessive redness or sunburn of the skin. In order to lessen redness or sunburn of the skin, some tanning compositions include conventional sun protection “sun blockers”, which filter out the ultraviolet component of the sun rays. These sun blockers in the tanning composition offer some protection against erythemal reaction in the skin. It prevents quick tanning where considerable portion of the ultraviolet lights are filtered out by these ingredients. The skin won't tan faster or deeper by the application of such compositions.

Erythrulose (D-Erythrulose): is another sunless tanning spray. It is a tetrose carbohydrate with the chemical formula C₄H₈O₄ which is very similar to DHA. Erythrulose is manufactured from the aerobic fermentation of the bacterium Gluconobacter followed by multi-step purification. It is a natural based keto sugar which reacts with the amino acid keratin. a protein on the outer or dead surface layer of the skin (the stratum corneum 15-40 layer of the epidermis). Like DHA, it does not involve melanocytes (skin pigmentation cells); nor does it require exposure to ultraviolet light to initiate the color change.

Tyrosine-based products: Although, gels, lotions, or sprays, that contain DHA and/or Erythrulose, are said to be the most reliable and useful; there are other types of products on the market. Tanning accelerators—lotions or pills contain the amino acid tyrosine that stimulate and increase melanin formation, thereby, accelerating the tanning process. These are used in conjunction with UVL exposure. At this time, there is no scientific data available to support these claims.

Canthaxanthin: This sunless tanning oral pill is used as a color additive in certain foods. FDA has approved the use of canthaxanthin in food, but not as tanning agent. After canthaxanthin is consumed, it is deposited throughout the body, including in the layer of fat below the skin, which turns into an orange-brown color. These types of tanning pills have been linked to various side effects, including hepatitis, canthaxanthin retinopathy, damage to the digestive system, and skin surface. The FDA withdrew approval for use of canthaxanthin as a tanning agent. The FDA has issued warnings concerning its use.

Melanotan hormone: Afamelanotide, a synthetic melanocytes-stimulating hormone analog, is approximately 1,000 times more potent than natural α-MSH. It induces melanogenesis through activation of the melanocortin 1 receptor. It is another tanning therapeutic agent considered. In 1991 clinical investigation of a new drug was conducted at the Department of Internal Medicine, University of Arizona Health Sciences Center with afamelanotide. Human skin darkens as a response to a synthetic melanotropin given by subcutaneous injection. Skin tanning develops without exposure to ultraviolet radiation. Using this method, tanning therapeutic agents can reduce skin cancer rates in people which would induce the body's natural pigmentary system to produce a protective tan prior to UV exposure. It may be implanted for continuous delivery (Clinuvel Pharmaceuticals of Australia is testing this method), for a series of afflictions affecting the skin such as erythropoietic protoporphyria (EPP), polymorphous light eruption (PMLE), solar urticaria (SU), photo toxicity associated with systemic photodynamic therapy and actinic keratosis (AK), squamous cell carcinoma, and skin cancer in patients whom have received organ transplants. This therapeutic agent will have wide therapeutic applications in dermatology to improve the tan and to protect the skin. Afamelanotide in a subcutaneous implant form is currently undergoing clinical trials and being developed by a company in Australia. I do believe this hormone with our invention and exposure of short periods of UVR can accelerate the development of long lasting tan and the depth of color.

Temporary bronzers: Bronzers are a temporary sunless tanning. The bronzing option is used in the form of powders, sprays, mousse, gels, lotions, and moisturizers. After application, they create a tan that can easily be removed with soap and water. These products tint or stain your skin like make-up, until, they are washed off. They are often used for a “one-day” tan to complement a DHA based sunless tan. These products are safer than tanning beds, but the color produced can sometimes look orangey and splotchy if applied incorrectly. Many formulations are available that can cause visible rub-off onto snug clothing, especially neck collars.

A new trend is that of lotions or moisturizers containing a gradual tanning agent. A slight increase in color is observable after the first use; the color will continue to darken the more the product is used.

Air Brush tanning is a spray on tan done by a professional. It can last five to ten days fading with every shower. It is used for special occasions or to get a quick dark tan for shows and before wedding or proms. At-home airbrush tanning kits and aerosol mists are now available.

Structure of the Skin

It is important to know the structure of the skin to understand how various tanning methods change the skin color to various shades of brown to bronze melanin pigmentation called a tan. This is described in detail by Costin and Hearing which are incorporated here in (Gertrude E. Costin and Vincent J. Hearing. Human skin pigmentation: melanocytes modulate skin color in response to stress. The FASEB journal. 2007; 21: 976-994). The skin is an envelope wrapped around our bodies like paper around a parcel. It is one of our most versatile organs. Even with our ingenious modern machinery, we can't create a tough and highly elastic fabric that will withstand heat and cold, wet and drought, acid and alkali, microbial invasion, and the wear and tear of three score years and ten. The skin can repair itself, even, present a seasonable protection of pigment against the sun's rays. The skin is the most superb fighting tissue. Skin is a regulator of the body's temperature: an excretory organ capable of relieving the kidneys in time of need. It is a storehouse for chlorides. The skin is the factory for anthracitic vitamin D (ergosterol) formed by the action of the ultraviolet B rays of the sun on the sterols in the skin and Ergosterolis necessary for the mineralization of bones and teeth.

Skin is the most extensive and varied of the sense organs. In an average adult man the skin covers a body surface of 1.7 sq. meters (20 square feet). This accounts for about 15 percent of adult body weight weighing 6-7 pounds. Each hand has 17,000 tactile receptors and 1,300 nerve endings per square inch. The Skin or cutis (L. cutis=skin) is made up of two parts (FIG. 1.2): (1) the dermis (Gk. derma=skin) and (2) the epidermis.

The epidermis is composed of 5 layers and the top layers are continually replaced as new cells. The new cells are produced in the bottom layer will mature and are pushed to the surface. It is ectodermal in origin which composed of several distinct cell populations that are the keratinocytes and melanocytes (mixed with immune system cell population). These are the main constituents which the first comprise 95% of the epidermis. Melanocytes in epidermis, eyes, brain, and nails are derived embryologically from the neural crest which they are ectodermal in origin. These new cells replace old, dead cells, which wash off or brush off from the surface of the epidermis. The entire epidermis is replaced just about every 27 days once. We shed approximately 40 pounds (19 kg.) of dead skin cells in the course of our lifetime. The Epidermis is a non-vascular stratified epithelium of ectodermal origin with thickness between 5-100- to 600 μm (thickest on palm and soles) (Elias, P. M. (2005) Stratum corneum defensive functions: an integrated view. J. Invest. Dermatol. 125, 183-200).

The deeper layer of epidermis is living. It consists of several strata of polyhedral cells resting on a single stratum of columnar basal cells. The superficial layer, the horny layer or stratum corneum, is dead. It consists of 15-40 layers of dry, flattened, dead, scaly corneocytes cells without visible nuclei. They are filled with a protein keratin and a lipid to make an effective barrier. The surface cells are perpetually being rubbed away and are continuously replaced by cells of the germinative basal layer. Finger-like processes of the dermis, called papillae, protrude into the epidermis anchoring it. The layers of epidermis beginning with the outermost layer are: Corneum, lucidum (only in feet), granulosum, spinosum, and basale.

The stratum basale consists of basal keratinocytes which have stem cell-like properties. Two different types of neural crest-derived cells are found in this layer. They are Merkel cells (neuroendocrine cells responsible for the transmission of touch sensation through the cutaneous nerves) and the melanocytes that produce skin and hair pigment. Cells are formed through mitosis at the basale layer. The daughter cells move up the strata and change shape and composition when they die due to isolation from their blood source. The cytoplasm is released and the protein keratin is inserted. The cells eventually reach the corneum and slough off (desquamation). This process is called keratinization takes place within about 27 days. This keratinized layer of skin is responsible for keeping water in the body and keeping other harmful chemicals and pathogens from entering the body.

The skin is a natural barrier to infection. The outermost layer of epidermis may consist of 15 to 40 layers of these dead cells. From this layer, an estimated 30,000 cells are lost (shed) from the skin surface every minute. Epidermis also contains DNA repair enzymes, which help to reverse UVL damage. People who lack the genes for these enzymes suffer high rates of skin cancer.

Stratum spinosum contains irregular polyhedral keratinocytes with some limited capacity for cell division. There are the bone marrow-derived sentinel cells of the immune system called Langerhans cells which represent the antigen-presenting cells of the skin. They also play a vital role in immunological reactions like allergic contact dermatitis.

Stratum granulosum contains flattened, polyhedral non dividing keratinocytes producing granules of a protein called keratinohyalin. These granules increase in size and number as the cell nuclei gradually degenerate and the cells die. These cells flatten as dividing cells underneath them progressively push them toward the skin surface.

The stratum lucidum is composed of several layers of clear transparent cells (hence the name) which the nuclei are indistinct or absent. This layer is comprised of flattened, dead, keratinized cells that form a barrier to the external environment.

Stratum corneum contains nonviable, biochemically active cells called corneocytes. The keratinocytes continue to differentiate as they move from the basal layer to the stratum corneum. The result is cornified cells that contain abundant keratin and lack cytoplasmic organelles. It is these cornified cells that provide a barrier against the physical and chemical agents in the environment. They reduce transepidermal water loss from within to prevent invasion by infectious agents and noxious substances from the outside.

The Dermis or Corium is of mesodermal origin which it is made of bundles of collagen and elastic fibers. Its spaces contain pellets of fat, hair follicles, sweat glands, and sebaceous glands. It is generally 1 to 2 mm. thick, but is thicker on the palms and soles (4 mm or more). The dermis is thinner on the eyelids and external genital organs. In animals, when the Dermis is tanned, it is called leather. Skin gets 9% of the blood pumped from the heart that amounts to 400 ml/minute. During skin tanning, it can increase to 2000 ml/minute or more due to blood vessel dilatation by the UV rays heating the skin.

The dermis is a thick layer of connective tissue (collagen, elastic, and reticular fibers) with fibroblasts and accommodates the neural, vascular, lymphatic, macrophages, mast cells, and secretory apparatus of the skin (FIG. 1). The main cell type, fibroblasts, is required for synthesis and degradation of the extracellular matrix, excretory, and secretory glands (sebaceous, eccrine, and apocrine).

Sebaceous glands secrete triglyceride and cholesterol-rich sebum that lubricate the skin and keep supple and waterproof. The sebum contains glyceride/free fatty acids, wax esters, squalene, esters, and cholesterol. The hair follicle provides a protective niche to several stem cell populations in the skin, including keratinocyte stem cells, melanocytes stem cells, a population of epidermal neural crest stem cells, and the dermal stem cell compartment, known as the dermal papilla which reside in the dermis (Ito, M., Liu, Y., Yang, Z., Nguyen, J., Liang, F., Morris, R. J., Cotsarelis, G. (2005) Stem cells in the hair follicle bulge to contribute to wound repair without homeostasis of the epidermis. Nat. Med. 11, 1351-1354). These stem cells are necessary during wound healing. Sensory nerve receptors of Merkel and Meissner's corpuscles (for touch), Pacinian corpuscles (for pressure), Ruffini and Krause corpuscles (mechano-receptors-heat and cold sensation) are located in the epidermis and the dermis interfaced and located below the basal layer.

Skin pigmentation is due to two important actions: the synthesis of melanin (melanosomes) by melanocytes and the transfer of melanosomes to surrounding keratinocytes (Fitzpatrick, T. B., Szabo, G. (1959). The melanocyte: cytology and cytochemistry. J. Invest. Dermatol. 32, 197-209). The number of melanocytes in human skin of all types is essentially constant, the number, size, and manner which melanosomes are distributed within keratinocytes vary. The melanin content of human melanocytes is heterogeneous between different skin types and between different sites of the skin from the same individual which is regulated gene expression.

In general, highly pigmented skin contains numerous melanosomal particles. Lighter pigmentation is associated with smaller and less dense melanosomes. These distinct patterns of melanosome type and distribution are present at birth. They are not determined by external factors (such as sun exposure). The variation in skin and hair color among various races is determined largely by the number, melanin content, and distribution of melanosomes produced and transferred by each melanocyte to a cluster of keratinocytes surrounding it. When melanin is produced and distributed properly in the skin, dividing cells in the basal germinating cells are protected at least in part from mutations that might be caused by harmful UVR. They are responsible for the wide variety of skin complexions as described in table 1.

In addition to keratinocytes, fibroblasts, and possibly other cells in the skin produce cytokines, growth factors, and inflammatory mediators that can increase melanin production and/or stimulate melanin transfer to keratinocytes by melanocytes. Melanocytes growth factors affect the growth and pigmentation of melanocytes. The growth factors also affect the shape, dendricity, adhesion to matrix proteins, and mobility.

Melanin is not a static substance which is the reason our skin changes color in response to various stimuli. Our melanocytes cells can produce more melanin if stimulated by the sun, medications, or certain diseases. The clearest example is tanning occurs when our skin produces more melanin after sun exposure. Our skin may darken in response to certain drugs such as minocycline which is commonly used to treat acne or in response to certain medical conditions such as Addison's disease (see “Melanin and Medicine,” page 14-15), Our skin can produce less pigmentation or have lightened areas after a burn or other injury.

Ultraviolet Rays (UVR) Effects on Skin Tanning

The skin responds to UVR exposure by developing two defensive barriers: thickening of the stratum corneum and the elaboration of a melanin filter in cells of the epidermis. The mitotic rate of basal keratinocytes increases a day after UV exposure, reaches a maximum 2 days later, and maintains this level for 1 week. It declines and the skin regains the original thickness after 1-2 months if there has been no subsequent exposure. After UVR, the epidermal melanin unit responds with increased levels of tyrosinase (TYR) activity, increased synthesis of melanosomes (tanning and coloring pigments of skin and hair), and higher rates of melanosome transfer to keratinocytes to meet the new demand for melanosomes which are created by the proliferation of keratinocytes.

The known beneficial effect of UVB is the stimulation of vitamin D synthesis in the epidermis. One role of melanin in the skin is to neutralize the ROS generated by a variety of factors, including UVB that functions like a natural sunscreen. The public assumes that dark skin is UVR resistant and not adversely affected by UVR. On the contrary, the studies showed that even the darkest UV resistant skin types accumulate significant DNA damage (Tadokoro, T., Kobayashi, N., Smudzka, B. Z., Ito, S., Wakamatsu, K., Yamaguchi, Y., Korossy, K. S., Miller, S. A., Beer, J. Z., Hearing, V. J. (2003) UV-induced DNA damage and melanin content in human skin differing in racial/ethnic origin. FASEB J. 17, 1177-1179). These authors established that even very low UV exposures cause measurable damage to DNA in all types of skin (Darkest to pale skin). It was clear that the most severe DNA damage was in lightly pigmented skin.

In humans, an increase of skin pigmentation called tanning is physiologically stimulated by UVR. The skin darkening involves an increase in the number of melanocytes as well as stimulation of melanin synthesis and melanocytes dendricity. The melanocytes dendricity is a crucial morphological feature required for melanin transfer to keratinocytes. The tanning response has been shown to have two distinct phases termed:

a. Immediate pigment darkening; and

b. Delayed tanning.

Both have strong genetic determinants which are generally more pronounced in individuals with dark baseline (constitutive) pigmentation. Quick and transient brownish tan follows due to exposure of skin to UVA or visible light.

Aging results in a decline in functional melanocytes in both the skin and hair. Studies indicate that the number of functioning DOPA-positive melanocytes in non exposed human skin decreases with age by 8-20% of the surviving population each decade. Nevertheless, in UV-irradiated skin there are approximately twice as many melanocytes in unexposed areas, with a comparable decrease in melanocytes with age.

It is surprising that unlike hair color, there is no loss of skin pigmentation with age. In fact, the chronically sun-exposed skin of an older person is usually more pigmented than that of a younger subject of similar complexion despite the decreased melanocytes density in the former. This paradox has been explained by the greater functional activity in older melanocytes after many years of cumulative sun exposure (Gilchrest, B. A., Frederick, B. B., Szabo, G. (1979) Effects of aging and chronic sun exposure on melanocytes in human skin. J. Invest. Dermatol. 73, 141-143). In melanosomes, three enzymes are required to synthesize different types of melanin. Tyrosinase is responsible for the criticl steps of melanogenesis (including the rate-limiting initial step of tyrosine hydroxylation), tyrosinase-related protein (TYRP1) and DOPAchrome, tautomerase (DCT) are involved in modifying the melanin into different types. An interesting finding recently reported that the DCT is not expressed by melanocytes of human hair compared to human skin. This could potentially contribute to the premature loss of melanin (premature graying) production by functional melanocytes in human hair with age (graying hair and not changing in color due to tanning like skin) due to added cytotoxic stress of melanogenesis in the absence of DCT involved in modifying the melanin into different types (Commo, S., Gaillard, O., Thibaut, S., Bernard, B. A. (2004) Absence of TRP-2 in melanogenic melanocytes of human hair. Pigment Cell Res. 17, 488-497).

Delayed tanning gives rise to a much durable tan induced by repeated exposure mainly to UVB and to UVA or to visible light. It is a gradual process in which the skin starts darkening 48-72 hour after irradiation and reaches a maximum by 3 weeks after exposure. Such skin does not return to its original melanin content until 8-10 months later. Delayed tanning is dependent on both qualitative and quantitative changes within melanocytes, which enlarge in size, increase their dendricity, and develop a diffuse distribution of thick filaments in their cell bodies. Ribosomes, ER, and Golgi apparatus are more prominent, reflecting an increase in the synthesis of TYR, and melanosomes in all developmental stages, in their melanization, and in the number that are transferred to keratinocytes. Therefore, delayed tanning is due to an increase in melanocytes numbers and melanogenesis.

The dermis is the layer of skin beneath the epidermis that consists of connective tissue, cushions the body and skin from stress, and strain. The dermis is tightly connected to the epidermis by a basement membrane (basal lamina). The skin or cutis has four appendages: hairs, nails, sweat glands, and sebaceous glands. It contains the hair follicles, sweat glands, sebaceous glands, apocrine glands, and blood vessels. The blood vessels in the dermis provide nourishment and waste removal to its own cells as well as the Stratum basale of the epidermis. The dermis is structurally divided into two areas: A superficial area adjacent to the epidermis, called the papillary region (finger-like projections) and a deep, thicker area known as the reticular region.

The hypodermis (subcutaneous tissue) is not part of the skin, and lies immediately below the dermis. Its purpose is to attach the skin to underlying bone and muscle as well as supplying it with blood vessels and nerves. It consists of loose connective tissue, elastin, fibroblasts, macrophages, and adipocytes. The hypodermis contains 50 percent of human body fat. Fat serves as storing energy and acts as padding and insulation for the body.

Microorganisms like Staphylococcus epidermidis colonize the skin surface. The density of skin flora depends on the region of the skin. The disinfected skin surface gets recolonized from bacteria residing in the deeper areas of the hair follicle, gut, and urogenital openings. Oily surfaces like the face may contain as many as 500 million bacteria per square inch. Despite these vast quantities, all of the bacteria found on the skin's surface would fit into a volume the size of a pea.

The skin has many functions. The first is protection. The skin acts as an anatomical and histological barrier between the internal and external environment in bodily defense. Langerhans cells in the skin are part of the adaptive immune system. It plays a role in sensation, heat regulation, control of evaporation, aesthetics, and communication with the CNS when the skin is exposed to the environmental changes. It acts as storage for fat and synthesis of vitamins B and D. This synthesis is linked to pigmentation, with darker skin producing more vitamin B than D, and vice versa. The skin also plays a role in excretion by sweating and absorption (Oxygen, nitrogen, and carbon dioxide can diffuse into the epidermis in small amounts).

All the above functions are possible due to skin structure which makes the skin. An inch of skin has: 65 hairs, 9,500,000 cells, 95 to 100 sebaceous (oil) glands, 19 yards (17 meters) of blood vessels (20 Blood vessels), 650 sweat glands, 78 yards (70 meters) of nerves, 78 sensory apparatuses for heat, 19,500 sensory cells at the ends of nerve fibers, 1,300 nerve endings to record pain, 160 to 165 pressure apparatuses for the perception of tactile stimuli. The skin has 60,000 melanocytes and 13 sensory apparatuses for cold (Adapted from Joel Gerson, Milady's Standard Textbook for Professional Estheticians, 8th edition).

Another function of skin is aesthetic. Often, however, attractiveness based on external features (beauty is “skin deep”) has been overemphasized versus the importance of internal character. Tanning is one cosmetic aesthetic used all over the western culture, especially, by Caucasian females.

In different parts of the world, humans have highly visible differences in skin pigmentation due to melanin pigment in the epidermis. Individuals from African ancestry have a tendency to be darker with the skin color. The Northern European descents have paler skin. Between these two extremes are individuals from Asian, South-East Asian, Native Americans, Middle Eastern, Polynesian, and Melanesian descents whose skin is of a different color. It is the individual with the paler skin who seeks tanning more than any other group.

Overexposure to ultraviolet radiation is known to cause skin cancer (melanoma, squamous and basal cell carcinoma), make skin age and wrinkle faster, mutate DNA, and reduce or inhibit the immune system. Frequent tanning bed use increases the risk (three times) of developing melanoma which is the deadliest form of skin cancer. Overexposure to ultraviolet radiation induces at least two common genetic mutations. While DNA repair enzymes can fix some mutations which they are not sufficiently effective. This was demonstrated by the relation to cancer, aging, and other types of persistent mutation and cell death. For example, squamous cell carcinoma (a type of skin cancer) is caused by a UVB induced mutation in the p53 gene. Most aging of skin is due to UVA rays destroying collagen and connective tissue beneath the superficial layer of the skin. UVB rays do not reach as far as the UVA rays.

Excessive exposure to UVA radiation has risks which may cause premature aging, including wrinkles, sunspots, and loss of skin elasticity. A 2009 Associated Press article stated, “International cancer experts have moved tanning beds and other sources of ultraviolet radiation into the top cancer risk category, deeming them as deadly as arsenic and mustard gas.” The Irish Health Minister in August 2009 said that she was considering outlawing the tanning bed industry completely given that they are dangerous and are hugely contributing to people developing skin cancer. Tanning ages the skin prematurely, causing age spots, saggy skin, and wrinkles besides predisposing to skin cancers. Tanning beds are machines installed indoors that give off long wave ultraviolet light or UVA that are stronger than the ordinary shortwave (or UVB) rays emitted by natural outdoor light. The choice to get darker skin with the help of a tanning bed means getting your tan in a way that is faster and more convenient.

Tanning Methods

Tanning involves coloring: 1. the epidermal skin layers, mostly stratum corneum and/or 2. Stimulating the melanin producing cells in the epidermis by using melanin precursor and/or make more melanin by stimulating the melanocytes to UVA radiation and melanocytes stimulating hormone. The sunless tanning effect is due to chemical reaction of the tanning cosmetic agents such as DHA and Erytbrulose with dead stratum corneum cells. Staining the stratum corneum is temporary because it is shed every day from top to bottom, thus reducing the tanning effects. Exposing to bright sun light or UVA tanning lamps does produce long lasting tanning compared to sunless tanning. But the UV light produces irreparable skin damages with the possibility of cancer if used repeatedly.

Our invention causes tanning by combination of both mechanisms. Melanocytes make melanin all the time, but this may not be enough to satisfy the fair-skinned individual. Ultraviolet radiation causes one to produce more melanin and produce a tan. Skin makes the melanin to absorb the ultraviolet radiation and prevents it from damaging DNA of skin cells. To make melanin, melanocytes need the enzyme tyrosinase and the non essential amino acid tyrosine. Tyrosinase gene makes tyrosinase. Tyrosine is abundant because, most Tyrosinase converts the tyrosine to DOPA and later to Dopaquinone. The Dopaquinone forms black-brown eumelanin or red-yellow pheomelanin. Irish descents have melanocytes that make mostly the red-yellow pheomelanin form of melanin which doesn't absorb UV radiation and doesn't make skin darker with tanning. Melanin stimulating hormone (MSH) flows through the bloodstream from the pituitary gland and reaches the melanocytes which it encourages them to produce more melanin. If someone were to be injected with MSH, it would make more melanin. Applying lotions and swallowing pills can't make the melanocytes produce melanin. The UV radiation, melanin precursors, and melatonin hormone can induce the melanocytes to produce melanin.

Pigmentation is highly heritable, regulated by genetic, environmental, and endocrine factors that modulate the amount, type, and distribution of melanin in the skin, hair, and eyes. Its role in defining ethnicity which melanin plays an essential role in defending the body against harmful UV rays and other environmental challenges. The melanocytes transfer melanosomes through their dendrites to keratinocytes, where they form the melanin caps that reduce UV-induced DNA damage in human epidermis.

Skin reacts to UV radiation based on the number of melanin producing cells and production of melanin in the epidermis that is responsible for skin color. People want to produce artificially called tanning for cosmetic purposes.

TABLE I

| | Effects of UV radiation exposure of |
|---|---|
| Skin types | the skin |
| Pale skin and Freckled pale | Always burns, never tans well due to |
| skin like Irish | lack or paucity of Melanin production |
| | by melanocytes. |
| Fair skin, a bit below pale | Usually burns, light tanning after |
| skin | long exposure |
| Light Brown skin | UVR exposure produces good tan. |
| Medium Olive brown skin | Seldom burns unless exposed for |
| | long time, always tans well |
| Deep Brown skin | Tans skin well, making it much |
| | darker and long lasting. |
| Black skin | Has natural pigmentation; tans much |
| | darker with Sun exposure. |

Many people still think that ‘light’ skin is very different from ‘dark’ skin, in spite of the scientific evidence that shows biological skin has structural similarity among all the people of the world. Complicated studies of human skin pigmentation using special stains and the electron microscopic studies have shown that the differences in skin pigmentation among the so-called races of mankind are only very minor. A world-renowned authority in clinical dermatology, Dr Anthony du Vivier at London's King's College Hospital, accurately sums up present scientific knowledge on the subject: “There are the same number of melanocytes [melanin pigment-forming cells] to be found in both Negroid and Caucasian skin” (Anthony du Vivier, Atlas of Clinical Dermatology, Gower Medical Publishing Ltd, London, 1986, p. 23.2.).

All skin types have the same number of melanocytes whether black, brown or white. Other specialist in the field of skin research agree, that the differences in coloration arise from the way in which melanin (the dark pigment found in the skin of all people) is packaged. The melanosomes (tiny melanin-packaging units in melanocytes) are slightly larger and more numerous per cell in dark-skinned than light-skinned people. They don't degrade as readily which the melanosomes disperse into adjacent skin cells to a higher degree (Walter F. Lever, and G. Schaumberg-Lever, Histopathology of the Skin, 7th edition, J.B. Lippincott Co., Philadelphia, 1990, pp. 18-20). This means that the differences are at a sub cellular level. There are minor variations in very minute areas, called organelles that reside in the pigment cells. These variations are under the control of the normal principles of genetics.

In fact, the skin and hair color results from the relative levels of two types of melanin pigment: the dark-brown pigment discussed above, and a reddish version of pigment. All people produce the red pigment, but red-headed people lack the ability to produce normal levels of the dark pigment. This is thought to be due to a mutation in one of the genes involved in pigment manufacture in the skin cells (Philip Cohen, ‘Redheads come out of the shade’, New Scientist, 30 September, 1995, p. 18). It is important to note that the red-heads lack the ability to produce the protective dark pigment. Their red pigment reacts directly with sunlight to produce chemicals (reactive oxygen species) which cause damage to active dividing skin cell DNA. This leads to skin cancer. Red-heads must be doubly careful in their exposure to the sun. Red hair may be beautiful, but the condition almost certainly arose from a mutation causing the loss of ability to produce dark pigment.

Increased skin ‘aging’ and vulnerability to various types of skin cancer are among the disadvantages for Caucasians, due to their decreased amount of melanin in skin cells, which protects the DNA of the dividing epidermal cells by blocking UVR. The scientists at the FDA have calculated the number of minutes that it takes to produce a minimal erythema dose (the point where the skin is burned enough to turn pink). The most sensitive pale skin burned after 14 minutes of exposure which the most resistant dark skin took more than 100 minutes to burn. This is why black skinned people moving to Europe suffer from vitamin D deficiency. The small amounts of UVR in these climates are absorbed by the melanocytes which means that the vitamin D can't be produced to meet the physiological needs of the body.

Moles (nevi) are common skin lesions which they are due to a proliferation of the pigment cells, melanocytes. If they are brown or black in color, they may also be called pigmented nevi. Moles are benign in nature (harmless), but a malignant melanoma (cancerous mole) may arise within a mole. Nevi may form from other skin cells (e.g. vascular nevi are formed from blood vessels). Those derived from melanocytes are known as moles. Moles may be flat or protruding and vary in color from pink or flesh tones to dark brown or black assuming round, oval, or unusual shapes. They range in size from a couple of millimeters to several centimeters in diameter. Their number varies from 20 to 100 on the skin that may be present at birth. Exposure to sunlight increases the number and size of the moles. Teenagers and young adults tend to have the greatest number of moles. There are fewer in the elderly because some of them slowly fade away.

There is close relation between skin condition namely vitiligo and melanocytes cells. When melanocytes are destroyed in any part of the skin, there is no coloring substance melanin; the pigmentation has less spots of vitiligo which appears at the skin site as white spots. Melanocytes, the cells responsible for skin pigmentation, die or are unable to function in this condition. Vitiligo is an autoimmune disease and several genes have been associated with vitiligo that would encourage the immune system to attack melanocytes. Most of those genes are in the immune system. Some genes are in the melanocytes themselves. The incidence of vitiligo worldwide is less than 1%.

Melanin is important for sharpness of vision, melanin in the iris, choroid, and pigmented layer of retina serves to minimalize the number of light beams that enter the eye. It provides for the absorption of scattered light within the eye. In this way, pigmentation allows for more keen sight.

Poliosis is the decrease or absence of melanin (or color) in head hair, eyebrows, or eyelashes. It can give rise to a “Mallen Streak” that can be hereditary. This condition can cause white patches (singular or multiple—usually single) on the scalp or other parts of hairy areas. Most people confuse these white patches as simple birth marks. Poliosis can occur in healthy people which may represent no more than an anomaly of hair and skin pigmentation. It is observed in association with a wide variety of conditions. At the mild end of the spectrum, a minor genetic defect called piebaldism results in poliosis. Somewhat more significant, poliosis may be associated with pigmentary disturbances of the eye, as well as hair loss in hypogonadism and thyroid diseases. Localized changes in the skin may also cause poliosis. Nevi and various kinds of nevi and focal skin cancers may result in patches of white hair growth in the areas of affected skin. Poliosis can be found associated with several genetic disorders such as Marfan's syndrome, Vogt-Koyanagi-Harada (VKH) syndrome, and Waardenburg's syndrome. These syndromes involve other physical symptoms and mental retardation to varying degrees.

Melanin is the dark pigment of the hair, skin, the covering of the eye, hairs, eyes, and a part of the brain (substantia nigra). Melanin can be found in some tumors (like dark moles on the skin). It is made in the body from a portion of protein (the amino acid tyrosine). For your body to make the correct amount of melanin, one needs to have adequate protein in diet. Fruits contain very little protein. Good sources of protein include meat, fish, milk/milk products, eggs. Protein is found in varying amounts in grain food (bread, pasta, rice, noodles, etc.). Your body is programmed via your genes (your inheritance from your biologic parents) to produce a certain amount of melanin. That is the reason that we all have different colors to our skin. People who have darker skin simply have more melanin produced in their skins than people with lighter skins. Melanin does offer some protection from the damage that certain UV-wave lengths in sunlight can cause. Thus a darker-skinned person has more sun-protection than a fairer-skinned person. I don't think you can regulate the amount of melanin that your body produces naturally, unless you modify your genetic make-up somehow, and scientifically we aren't there yet.

Albinism is a condition marked by the absence of a normal amount of pigment in the body. Animals, humans, and even plants can have albinism. Albinism exists in a number of variations. Depending on the type of albinism, the skin, hair, and eyes may all be affected. In fact, ocular albinism affects not only the color of the eyes, hair, and skin which results in poor vision. Additionally, some types of melanin deficiency are associated with increased mortality rates.

Several recent patents have disclosed various skin tanning compositions. For example, U.S. Pat. No. 3,988,437 discloses a suntan composition which contains a fluorescent compound for protection against sunburn and to promote tanning.

U.S. Pat. No. 4,434,154 discloses a tanning and ultra violet screening composition. The composition comprises dihydroxyacetone (DHA), octyldimethyl PABA, water oil and surfactant. The surfactants are sodium alkyl sulfates wherein the alkyl group contains 8 to 16 carbon atoms.

U.S. Pat. No. 5,061,480 discloses a skin tanning and conditioning composition for accelerating the tanning process comprises specified nonionic surfactants together with tyrosine, protein hydrolysate and either riboflavin or adenosine triphosphate.

U.S. Pat. No. 7,537,584 B2 invention teaches devices and methods for chemically tanning human skin.

U.S. Patent Application Publication Number: 2004/0013617 A1 invention relates to a sunless tanning composition comprising at least one sunless tanning active ingredient, at least one phospholipid, at least one nonionic surfactant, and at least one amphoteric surfactant. The sunless tanning active ingredient can be dihydroxyacetone or erythrulose which provide enhanced tanning compared to a formula having the same level of sunless tanning active ingredient but without the phospholipid, nonionic surfactant, and amphoteric surfactant. The combination of phospholipid, nonionic surfactant, and amphoteric surfactant has been previously reported in U.S. Pat. Nos. 6,015,574 and 6,221,389, as a delivery system for lipophilic (oil-soluble) materials in an aqueous solution.

U.S. Pat. No. 4,714,609 disclose a method for tanning the human epidermis comprises a base or carrier and an effective amount of vanillin. The vanillin reacts with proteins in the human epidermis, when exposed to the sun's rays to accelerate tanning.

U.S. Patent Application Publication No.: 2007/0020202 A1 disclose one or more sunless tanning enhancers are selected from the group consisting of primary amines, oligomeric siloxane, amino acids, polyamines, amides, peptides, proteins, amphoglycinates, and any combinations thereof.

U.S. Patent Application Publication Number: 2009/0232750 A1 disclose inducing natural tan of the skin by using D-ribose 3-20% dissolved in water as an active ingredient; a penetrant; and a emollient such as mineral oil, a vegetal oil, an animal fat or an alcohol derivative of a vegetal oil or an animal fat, sodium lauryl sulfate, sodium stearyl lactate, glyceryl dilaurate, sorbitol and isopropyl myristate to deliver it to the basal cells of the dermis of the skin. It is said to act by stimulating the melanin production from the melanocytes by enhancing the cell activity by increased ATP production in all the cells including melanocytes that need to be applied for 5 days to obtain the desired results. Preliminary experiments showed that D-ribose applied directly to a wound caused the surface to become brown, possibly due to the Maillard reaction between D-ribose and the proteins in the wound. However, D-ribose in water applied to the skin showed no such effect on tanning. The object of using D-ribose is to increase the metabolic activity of dermal layers by enhancing the ATP production, thus increase the health of the skin at the same time increase melanin production.

U.S. Patent Application Publication Number: 5,425,784 disclose Water-soluble graft polymers of monosaccharides, oligosaccharides, polysaccharides and derivatives thereof, obtainable by free radical polymerization.

U.S. Pat. No. 4,781,914 discloses a sunscreen and moisturizer composition containing polyglycerol-8-Oleate for imparting moisture resistance or substantivity to the composition.

U.S. Pat. No. 4,783,332 discloses a skin tanning composition comprising various ingredients including tyrosine or tyrosine precursor to panthenol and ethoxylated glycerides esterified with fatty acids as described therein.

In a paper published in 1987, Dr. Christine Jaworsky was doubtful about the claims of many producers of sun tan' products who have claimed that their products contain active ingredients which accelerate the tanning process (American Academy of Dermatology, Vol. 16, 1987, page 34). There isn't a suitable tanning composition which satisfies the consumers' desire for quicker, deeper, long lasting and more intense tan with safety. It is the object of this invention to provide such tanning compositions which promote and accelerate tanning of the skin without the need for prolonged repeated exposure to harmful UVR rays once the desired tanning is achieved.

It is an objective of this invention to provide tanning composition which produces a deeper, intense, rapid and uniform tan on the skin. It is another object of this invention to produce durable long lasting tan to prevent the repeated exposure to harmful UVR. The foregoing and other advantageous features of the present invention will be more fully appreciated from the ensuing detailed description.

### SUMMARY OF THE INVENTION

The invention relates to skin tanning and conditioning composition which is capable of imparting intense, deep, safer, more uniform and long lasting durable tan than the currently available suntan products and methods.

The invention relates to Rifampin and its derivatives with cosmetic applications to produce durable UVR mediated tanning, which avoids repeated exposure to harmful UV rays.

It is therefore an object of this invention to provide a tanning composition and therapeutic agents which accelerates the skin tanning process.

### BRIEF DESCRIPTION OF THE DRAWINGS

FIG. 1 is the diagram of skin showing epidermis, dermis, superficial fascia, and deep fascia and the cellular layers which are involved in our inventions tanning methods.

FIG. 2 is the diagram of skin showing 5 epidermal layers and melanocytes that play a role using various tanning methods of the skin.

### DETAILED DESCRIPTION OF THE INVENTION

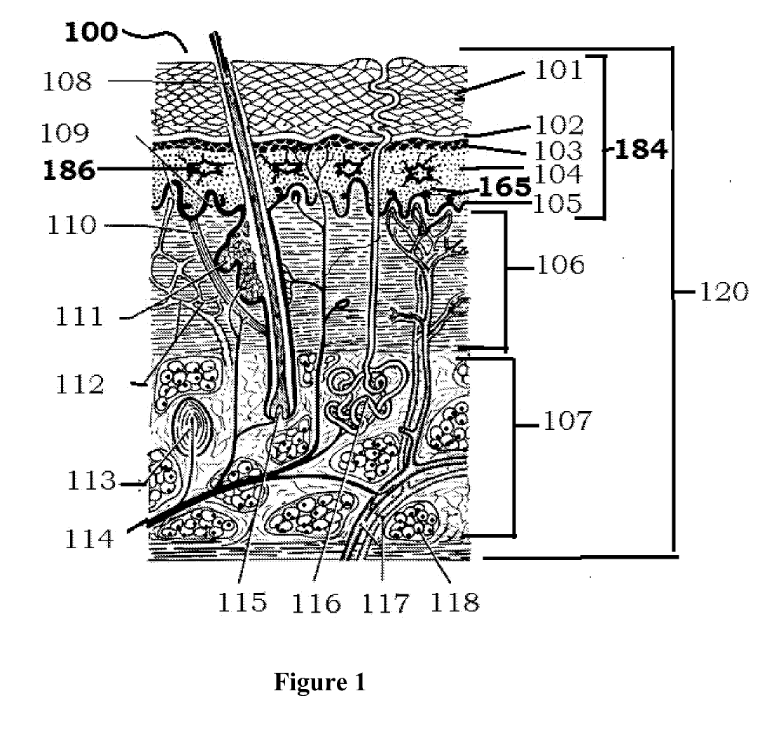

FIG. 1 is the diagram of skin 100 showing epidermis, dermis, superficial fascia, and deep fascia and the cellular layers which are involved in tanning methods. It shows Epidermis 184 made up of stratum corneum 101, stratum lucidum 102, stratum granulosum 103, germinative (stratum spinosum, spinous layer) layer 104 containing Langerhans dendritic cells 186 (located between stratum granulosum 103 and stratum basale 105), Basal layer of columnar cells 105 and melanin producing melanocytes 165, Dermis or Corium 106 with various components, Superficial fascia 107, Hair 108, Dermal Papillae 109, Arrector Pili smooth muscle 110, Sebaceous gland 111, Lymphatic plexus 112, Pacinian corpuscle 113, Nerves 114, Papilla of the hair 115, Sweat gland 116, Blood vessels 117, Fat in the subcutaneous tissue 118, Epidermis 184, Epidermis, Dermis, and subcutaneous tissue (superficial fascia) 120 situated on the deep fascia covering the muscles below it (modified from Grant).

Sunless tanning stains the cells in the stratum corneum 184, 284. Sun tanning provides skin tanning is due to melanin produced by the melanocytes and transferred to the keratinocytes of this epidermis 184 and 284. The melanocytes are involved in the production of the tan by producing melanin pigment when exposed to the sun UVR, melatonin hormone, melatonin releasing hormones, Afamelanotide hormones, and tyrosine as well as D-ribose. Rifampin in our inventions colors all the above described layers of the skin which possibly augments and amplifies the effects UVR to produce more melanin. The effect of this augmentation—amplification continues for a long period of time imparting most durable deep tan (modified from Grants method of Anatomy).

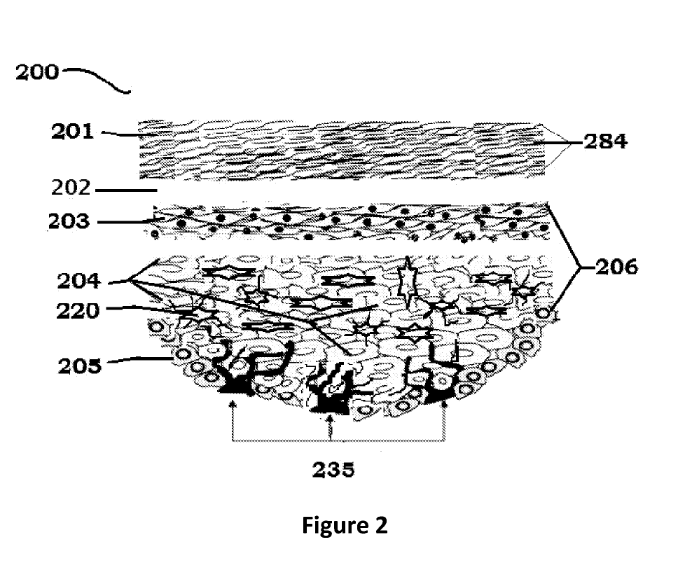

FIG. 2 is the diagram of the skin 200 showing epidermis situated on the dermis showing the cellular layers which are involved in tanning methods. It shows Epidermis and its various cellular components 206, 284, which are involved in the tanning methods. It shows multiple layers of stratum corneum 201, 284 which are stained by using sunless tanning solutions such as DHA and Erythrulose; stratum lucidum 202, stratum granulosum 203, multiple layers of germinative (stratum spinosum, spinous layer) layers 204, which are located on one layer of dividing basal layer of columnar epidermal cells 204, 205. Langerhans immune cells 220 embedded in the germinative layer involved in presenting the antigens to the immune system; Melanocytes 235 in the Basal layer of epidermis produce melanin pigment granules and transfer it to keratinocytes of the stratum germinative layer 204 which in turn imparts color to the skin of the humans. It is the amount and the concentration of melanin in the layer 220 and 206 responsible for different color of diverse racial groups of the world and the tanning effect of UVR, our invention, tyrosine, afamelanotide hormone. The Langerhans cells also known as dendritic cells in the epidermis are located in the stratum spinosum 186, 220 which are the major components of keratinocytes layer. These Langerhans cells contain large granules called Birbeck granules. They are derived from the cellular differentiation of monocytes with the marker “Gr-1” which can be found elsewhere in the body e.g. Lymph nodes. One of the important functions of these cells in the skin is to pick up (engulf) and to process microbial and other antigens foreign to our body. They become fully activated and function as antigen-presenting cells of the immune system.

Melanocytes 165, 235 play a major role in the skin color, tanning and skin cancers (melanomas). They are involved in the production of melanin mediated tan when exposed to Sun UVR, melanocyte stimulating hormone, Afamelanotide hormones, and tyrosine. Rifampin in our inventions colors all the above described layers of the skin, interstitial tissue, connective tissue, fat, and other cellular elements of the epidermis, dermis as well as subcutaneous tissue. It possibly augments and amplifies the effects UVR, to produce more melanin by melanocytes in the basal layer as we noted in our present invention. The effect of this augmentation—amplification by Rifampin continues for long period of time imparting long lasting most durable deep tan.

Bronzers are a temporary sunless tanning or bronzing options used in the form of powders, sprays, mousse, gels, lotions and moisturizers. They stick to the stratum corneum 101 and 201 of the epidermis after application that creates a tan (makeup of the skin) which can be easily removed with soap and water. Like make-up, these products tint or stain your skin only until they are washed off. They are often used for a “one-day” only tan, or to complement a DHA based sunless tan.

The tanning product in our invention is a Rifampin, a derivative of rifamycin. It is used all over the world to treat tuberculosis, leprosy, mycobacterium avium complex (MAC) and other infections. In combination with other antibacterial therapy, rifampin has many important applications in treating chronic deadly infections with good safety record when used for more than six months. The tuberculosis patients using Rifampin for many months became much darkly stained which was due its additional effect with exposure to the Sun light described in this invention. It exhibits good activity against many gram-positive cocci (including methicillin-resistant Staphylococcus aureus-MSRA), some gram-negative organisms, Legionella, many mycobacterium and some fungi.

Rifamycins were first isolated in 1957 from a fermentation culture of Streptomyces mediterranei at the laboratory of Gruppo Lepetit SpA in Milan by a scientist named Piero Sensi, working with the Israeli scientist Pinhas Margalith. Finally, seven rifamycin like compounds were discovered which they were called as Rifamycin A, B, C, D, E, S and SV. Of the various rifamycin families, Rifamycin B was first introduced commercially. Lepetit filed for patent protection of Rifamycin B in the UK in August 1958 and in the US in March 1959. The British patent GB921045 was granted in March 1963 and U.S. Pat. No. 3,150,046 was granted in September 1964 as antimicrobial but not as tanning enhancer. This drug is widely regarded as having helped to conquer the issue of drug-resistant tuberculosis in the 1960s. It is one of the main antibiotics used for 6 months or more, taken orally in 600 mg doses as a part of 4 drug treatment regimens to treat ravaging tuberculosis which affects and kills millions all over the world.

Rifamycins have been used for the treatment of many diseases which most importantly the HIV-related Tuberculosis. Due to the large number of available Rifampin analogues and derivatives, rifamycins have been widely utilized in the elimination of pathogenic bacteria that have become resistant to commonly used antibiotics. For instance, Rifampicin is known for its potent effect and ability to prevent drug resistance. It rapidly kills fast-dividing bacilli strains as well as “persisters” cells, which remain biologically inactive for long periods of time, that allow them to evade antibiotic activity (Calvori, C.; Frontali, L.; Leoni, L.; Tecce, G. (1965). “Effect of rifamycin on protein synthesis”. Nature 207 (995): 417). In addition, rifabutin and rifapentine have both been used against tuberculosis acquired in HIV-positive patients.

Derivatives of Rifampins:

Lepetit introduced Rifampicin, an orally active rifamycin, around 1966. Rifabutin, a derivative of rifamycin S, was invented around 1975 and came on to the US market in 1993. Hoechst Marion Roussel (now part of Aventis) introduced rifapentine in 1999. Rifaximin is an oral rifamycin marketed in the US by Salix Pharmaceuticals that is not absorbed from the intestine. It is intended to treat intestinal infections due to Escherichia coli, hence, cannot be effective for tanning.

Side Effects:

Taken orally or administered intravenously, the Rifampin discolors the tears, urine and feces. This is harmless. Hepatic enzyme induction may result in decreased levels of other medications (i.e., Coumadin, oral contraceptives, clarithromycin, and azoles) that are metabolized by the same pathway. Rifabutin (another rifamycin) exhibits less hepatic enzyme induction and less effect on other drug levels. Other side effects include gastrointestinal upset, rash, hepatotoxicity, hypersensitivity and a flu-like syndrome. This drug may cause stomach upset, heartburn, nausea, headache, drowsiness, or dizziness. These effects should disappear as a body adjusts to this therapeutic agent. Soft contact lenses may become discolored. In the unlikely event you have an allergic reaction to this drug; the patient needs to seek immediate medical attention. Symptoms of an allergic reaction include: rash, itching, swelling, dizziness, and difficulty with breathing. If you notice other effects not listed above, contact your doctor or pharmacist.

Precautions:

Tell your doctor your medical history especially if you have liver problems, history of alcohol use, or any allergies. Alcohol can reduce the effectiveness of this medication and increase the side effects. This medication shouldn't be used during pregnancy for tanning. Rifampin is excreted into breast milk. To date, the problems haven't been reported in nursing infants. Caution is a necessity if the tanner is breast feeding.

Drug Interactions:

This drug isn't recommended for use with delavirdine. Inform your doctor about all the medicines you may use (both prescription and nonprescription): warfarin, oral medicines for diabetes, azole antifungals (e.g., itraconazole, ketoconazole), theophylline, halothane, verapamil, certain protease inhibitors (e.g., nelfinavir), corticosteroids (e.g., prednisone), disopyramide, beta-blockers (e.g., metoprolol), isoniazid, digitoxin, quinidine, mexiletine, phenyloin, cyclosporine, zidovudine and live vaccines. Report drugs which cause drowsiness such as: sedatives, tranquilizers, psychiatric medicines, anti-seizure drugs, anti-anxiety drugs, narcotic pain relievers, antihistamines (e.g., diphenhydramine).

Rifampin can decrease the effectiveness of oral contraceptives. Do not start or stop any medicine without doctor and/or pharmacist approval. Rifabutin (another rifamycin) exhibits less hepatic enzyme induction and less effect on other drug levels.

Overdose:

Symptoms of overdose may include swelling of face or around eyes, itching over the entire body, orange or red discoloration of skin or eyes, nausea, vomiting, drowsiness, and loss of consciousness. If overdose is suspected, contact your local poison control center or emergency room immediately. One needs to take the prescribed amount for tanning to avoid any overdosing. At this time, we haven't noticed any overdose effects in our dose of using 600 mg to 1200 mg as a daily dose.

Rifampin, in medical practice is used at dosages of 10 to 20 mg per kg per day (intravenous or oral), with a maximal dosage of 600 mg per day. In pediatric suspensions it can be formulated in doses of 15 or 50 mg per ml in simple syrup which it is commercially available in 150, 300, 450 mg breakable capsules.

Rifampin is marketed and is used for bacterial infections as described above. None of them describe the use for tanning skin. Rifampin tanning effect was an accidental discovery, when a patient taking Rifampin went to the beaches in Turks and Cacaos. The person sunbathed 2-3 hours a day for 3 days. When he came back, his extremities, chest, and abdomen were tanned bronze brown. He went on thinking the tan will go away, but the color didn't fade even after 4 months and was fading very slowly. Then it was tried as experimental study on volunteers and tanning booth users. It gave the same results. It gave good natural looking long lasting tanning with minimum UVR exposure. The effects of tanning were more pronounced in brown skinned compared to light and white skinned people.

We know the safety of using Rifampin, which relates to its safety for tanning use. The rifamycins have been shown to have a very low toxicity for mammalian organisms and cells (Wehrli Walter, Staehelin Matthys. Actions of the Rifamycins. Bacteriological Reviews, September 1971, p. 290-309 Vol. 35, No. 3). The question arose whether this least biological effect on the mammalian cells is due to the fact that the rifamycins can't reach their site of action or they are ineffective against mammalian RNA polymerases? Studies with RNA polymerase from rat liver nuclei and ascites cells showed that they contained an enzyme insensitive to rifamycins. These results have been confirmed with solubilized RNA polymerase isolated from rat liver, human placenta nuclei, and lymphoid tissue. The activity of these enzymes depended completely on the addition of DNA. Various authors claim to have obtained enzyme inhibition in mammalian cells. The amounts of rifamycins used are 100 to 1,000 times higher than those needed to inhibit bacterial RNA polymerase and/or tanning. These studies indicate that under given doses to treat infection that has no or minimal effect on the host cells when used for tanning and used no more than a week.

Rifampin in doses of 600 mg to 1200 has been used by millions people all over the world for up to 6 months or more at each round of treatment for treating tuberculosis without any serious adverse effect to the body. It has one of the best safety records taken for such a long time. It can be safely used for tanning in the doses of 600 to 1200 mg (dose depending on the weight) day orally for 3-5 days or till one obtains the desired tan without any untoward effects.

The Mechanism of Skin Tanning by Rifampin and Other Cosmetic Agents

The mechanism of skin tanning by Rifampin isn't described in the literature. Rifampin action as skin tanning is based on the various metabolic and pharmacological changes that it undergoes after ingestion. Approximately 85% of rifampicin is metabolized by the liver microsomal enzymes that the main and active metabolite is deacetylrifampicin. Rifampicin undergoes enterohepatic recirculation but not the deacetylated form. Rifampicin may be inactivated in other parts of the body. Formylrifampicin is a urinary metabolite that spontaneously forms in the urine. Deacetylrifampicin from the liver enters into the circulation and exits the blood vessels to enter extracellular fluids (ECF) including the skin. From ECT, it enters the cells through the cell membrane into the intracellular fluid (ICE) and cell contents—organelle and nucleus.

It is important to note that with rare exceptions, the circulating blood doesn't come into direct contact with the cells it nourishes. As blood enters the capillaries surrounding a tissue space (ECR), a large fraction of it is filtered into the tissue space in the ECF filtrate. It is interstitial or extracellular fluid (ECF) that brings to cells all of their metabolic requirements and takes away their end products. It is the ECF content of the deacetylrifampicin which colors all the tissue of the skin in tanning as shown in FIGS. 1 and 2. The number and distribution of capillaries is where no cell is ever farther away than 50 μm from a capillary. As deacetylrifampicin enters ECT, cell wall, and ICF, it stains the interstitial tissue spaces. Its content, cell contents and cell membranes give the tanning appearance.

One of the important effects of sun bathing or tanning booth is increase in skin temperature exposed to UVR rays. Normal body temperature is 36.5° C. (98.5-99.5° F.). With sunbathing or under tanning booths, the skin temperature rises rapidly up to 100° F. to 100.5° F. With rise of temperature, the physiological effects, i.e. response of the skin to elevated temperature are profound. UV radiation produces hot, dry skin, a typical sign of skin hyperthermia. The skin becomes red and hot as 19 yards or 57 feet's (17 meters) of 20 Blood vessels per Sq. inch in the skin dilate. This increase in skin blood flow rate creates an arterialization (arteriovenous shunt) of arterio-venous junction and increases the oxygen tension in the capillaries and the surrounding tissue. This is the reason the skin looks red during tanning. The skins' inability to cool the body through perspiration causes the skin to feel dry.

Normally, the skin gets 9% of the blood from the heart that amounts to 400 ml/minute. Skin tanning due to UV radiation from the sun or other means can increase the blood supply 3-7 (from 400 to 2000 ml or more per minute) times more than resting skin. These results are more coloring with Rifampin and its metabolites to all the skin layers. Increased concentration in skin ECF and spaces of the epidermis, dermis, and subcutaneous tissue contents are immersed in this coloring metabolite which is seen as tanning. From the ECF of the dermis, the Rifampin permeates to the other layers of epidermis, reaching the top most layer-stratum corneum.

Besides this hyperthermia induced amplified blood supply, UVA radiation stimulates melanocytes (about 60,000 of them per square inch) of the skin to make more melanin pigment which is the natural coloring agent of the skin. Increased blood supply brings more nutrients and melatonin hormone to the skin melanocytes to augment the production of melanin. This increased blood flow will bring more ECF containing Rifampin. Its active metabolite deacetylrifampicin: to the epidermis, dermis, and subcutaneous tissue (mostly fat), nerve structures and all the other appendages of skin located below the epidermis. It is highly likely that the sebum producing glands and melanin granules gets stained by rifampin metabolites much more deeply due to their oily lipid proteins complex secretions which impart the color to the skin.

All the cells of the dermis and epidermis get this coloring Rifampin metabolite which colors these cells more than any other cells in the body. The Rifampin from the dermis ECF permeates the basal layer all the way to Stratum corneum coloring. The epidermis layers are completely bathed by rifampin as it permeates the skin layers. All the glandular system of the skin gets the Rifampin from the ECF Imparting the color to the epidermis. This is recognized in the sweat glands. They start producing copious amount of the sweat as a result of heat from exposure to UVR which also excretes the rifampin metabolites contributing the epidermal coloring. The coloration from the permeation of ECF loaded with deacetylrifampicin from the dermis and subcutaneous tissue which stains connective tissue and intercellular matrix made up of complex fat, carbohydrate, and protein complexes.

Many cellular components include the fibroblasts which contribute to collagen production. It is very likely that the Rifampin metabolites stimulate the production melanin pigment in the basal cell layer. Further, increased blood supply to the skin brings Melanocytes stimulating hormone to the basal layer of the epidermis which stimulates them to make more melanin. The melanin pigments get coated with Rifampin resulting in deeper coloration and stimulation of more coloring molecules. This result in durable, long lasting bronze like tanning by Rifampin; adding to the UV stimulated tanning. One of the added beneficial effects of using Rifampin for tanning is reduction of millions of bacteria hiding in the deep pockets of skin appendages such as sweat glands, sebaceous glands, and the hair follicles. The experimental test individuals using this method of tanning reported less dandruff production, which is probably due to antifungal and antibacterial effects of Rifampin on the scalp infection.

It is well known that the all the fluids in the body become yellowish bronze color after oral intake of Rifampin. The urine, faces, lacrimal fluid, and saliva all become colored by the distribution of the metabolite of the Rifampin. As the contact lens is made of plastic polymers, the users of Rifampin for tanning were asked to remove them during tanning because it is excreted into the tears which can color the contacts. It distributes to the skin ECF in large amounts compared to any other organ due to 3-5 times greater than before blood pouring as a result of heating induced vasodilatation of the skin due to UV radiation. Both UVR and the Rifampin play role in producing a durable tanning produced within a one-week session.

The dermis besides collagen and other secretory and excretory structures contains the complex extrafibrillar matrix. It is composed of a complex mixture of proteoglycans, glycoproteins, glycosaminoglycans, water, and hyaluronic acid. The glycosaminoglycans bind to proteins to form the proteoglycans of the skin to form chondroitin sulfate, dermatan sulfate, keratin sulfate, heparan sulfate, and heparin. Many of the proteoglycans of the skin are versican, involved in assuring the tightness of the skin, and perlecan, which is found in basement membranes. Glycoproteins, such as laminins, matrilins, fibronectin, fibronectin, tenascins, etc., are involved in cell adhesion, cell migration, and cell communication, which are extremely important processes taking place in the skin (Costin and Hearing IBID). The deacetylrifampicin of our invention from the extracellular fluid imparts color to these structures contributing enhancing and contributing to the tanning.

Method of Using Rifampin for Tanning

Rifampin is taken orally with water, one to two hours before going to the tanning booth, spas or before exposing to natural sun tan on decks, swimming pools and beeches. Orally-administered Rifampin results in peak plasma concentrations in about 1-2 hours. Apply moisturizers to the skin to prevent UVR induced drying. Expose the skin 2-4 hours a day to the sun. Continue for a week with this method. If tanning booths are used, do not expose more than indicated safe time that is given foe the tanning device.

After about 6 hours, almost the entire Rifampin is deacetylated. In this deacetylated form, Rifampin is still a potent antibiotic; however, it can no longer be reabsorbed by the intestines. It is, subsequently eliminated from the body through body fluids. Only about 7% of the administered drug will be excreted unchanged through the urine. The urinary elimination accounts for only about 30% of the dose of the drug that is excreted. About 60% to 65% is excreted through the feces. Approximately 50% of the rifampicin dose is eliminated within 24 hours.

The half-life of rifampin ranges from 1.5 to 5 hours, though hepatic impairment will significantly increase the drug. Food consumption inhibits absorption from the GI tract and the drug is more quickly eliminated. Hence, it should be taken 2 hours before or 2 hours after meal for tanning or for other therapeutic indications. When rifampin is taken with a meal, peak blood concentration is 36%. Avoid taking the medication with food. Antacids do not affect absorption. The urine color can be used as a marker. Whether or not a dose of the drug has been effectively absorbed from the intestines, the urine and facial orange coloration disappears within 24 hours of its use for tanning.

After a 450 mg oral dose, plasma levels reach 6 to 9 μg/mL while a 600 mg dose on an empty stomach yields 4 to 32 μg/mL (mean 7 μg/mL). The dose taken is 600 mg orally with water. Avoid alcohol after taking Rifampin for two hours. If the person is overweight, the dose should be increased to 900 to 1200 mg depending on the body weight.

It can be used with other tanning agents. If one desires immediate tanning the same day, use the combination of oral Rifampin and sunless tanning agents which contain DHA and Erythrulose.

Tyrosine (amino acid), in the form of gel or oral pill can be combined with Rifampin oral capsules to enhance the tanning effect. Tyrosine stimulates and increases melanin formation in melanocytes by tyrosinase (TYR) enzyme which accelerates the tanning process. These are used in conjunction with UV exposure.

Melanotan hormone, also, known as Afamelanotide, a synthetic melanocyte-stimulating hormone analog. This induces melanogenesis through activation of the melanocortin 1 receptor. It is another tanning therapeutic agent considered with Rifampin. This needs to be injected subcutaneously and takes many days to see the results. This method of skin tanning appears possible without potentially harmful exposure to ultraviolet radiation. Combining Rifampin and Melanotan hormone can produce rapid and lasting tanning effect.

Rifampin can be used with the composition comprises tyrosine as the active ingredient together with protein hydrolysate and riboflavin to avoid prolonged exposure to sun rays or other sources of ultra violet radiation and to produce the desired tanning effects. Tyrosine and its derivatives are capable of penetrating the deeper layer of the skin when applied percutaneously and causes tanning of the skin when exposed to the sunlight or UVR. Adding small amounts of riboflavin accelerates the oxidation process which produces the skin tanning pigment from tyrosine. In addition, riboflavin imparts a yellowish color to the resulting composition and skin with the use of these nutriceuticals.

Rifampin can be used with the non-ionic surfactants used in the compositions of this invention to promote and to accelerate the tanning process include polyoxyethylene 4 lauryl ether (Laureth 4) and sorbitan laurate, which are the preferred surfactants.

Rifampin can be used as a method for tanning the human epidermis along with Vanillin. The vanillin applied over the skin reacts with proteins in the human epidermis, when exposed to the sun's rays to accelerate tanning with Rifampin combination.

Rifampin can be used with a preparation of the tanning compositions comprises mixing water at a temperature of from about 20.0 to about 30.0 C., with an aqueous concentrate containing tyrosine (or a derivative thereof), protein hydrolysate, and riboflavin or adenosine triphosphate, and agitating the resulting mixture to obtain a homogeneous solution. The non-ionic surfactant is added to the solution and continued stirring. The final product has a smooth, creamy consistency. The composition of this invention comprises other conventionally employed ingredients such as a thickener, a biocide, cocoa butter, an emollient, and a pH adjuster. The rifampin can be used with these surface application preparations.

Rifampin can be used with a preparation of the tanning compositions comprises applying topically D-ribose with composition capable of carrying the D-ribose to the lower layers of the epidermis. The topical composition of D-ribose may be incorporated into a pad, spray, a lotion or a cream. The composition is applied daily, twice or thrice daily, to skin that is to be treated for tanning until the desired results are obtained. The D-Ribose at a concentration of 5% is dissolved in water plus 2-10% ethanol water v/v solution can be sprayed in from a spray bottle to the surface of the skin to be tanned. This tanning composition is applied in combination after oral intake of Rifampin as described above.

When using tanning body spray or mist in a commercial spray “tanning” booth, the areas of the eyes, lips, or mucous membrane, should be protected from exposure.

Numerous modifications and alternative arrangements of steps explained herein may be devised by those skilled in the art without departing from the spirit and scope of the present invention and the appended claims are intended to cover such modifications and arrangements. Thus, while the present invention has been described above with particularity and detail in connection that is presently deemed to be the most practical and preferred embodiments of the invention. It will be apparent to those of ordinary skills in the art that numerous modifications, including, but not limited to, variations in size, materials, shape, form function and manner of procedure, assembly and uses may be made.

While the preferred embodiment of the present invention has been described. It should be understood that various changes, adaptations and modifications may be made thereto. It should be understood, therefore, that the invention is not limited to details of the illustrated invention examples.

What is claimed is:

1. A method of tanning the skin comprising the step of administering a therapeutically effective dose of Rifampin.

2. The method of tanning the skin according to claim 1 further comprising the step of using at least one other tanning agent in conjunction with said dose of Rifampin.

3. The method of tanning the skin according to claim 1 further comprising the method of administering therapeutically effective doses of Rifamycin A, B, C, D, E, S and SV.

4. The method providing durable tanning the skin according to claim 2 wherein said at least one other tanning agent is Dihydroxyacetone (DHA).

5. The method of tanning the skin according to claim 2 wherein said at least one other tanning agent is Erytbrulose.

6. The method of tanning the skin according to claim 2 wherein said at least one other tanning agent is Tyrosine,

7. The method of tanning the skin according to claim wherein said at least one other tanning agent is an Afamelanotide

8. The method of tanning the skin according to claim 2 wherein said at least one other tanning agent is surfactants agent polyoxyethylene 4 lauryl ether (Laureth 4) and sorbitan laurate.

9. The method of tanning the skin according to claim 2 wherein said at least one other tanning agent is Vanillin.

10. The method of tanning the skin according to claim 2 wherein said at least one other tanning agent is selected from the group consisting of tyrosine, protein hydrolysate, riboflavin, and adenosine triphosphate.

11. The method of tanning the skin according to claim 2 wherein said at least one other tanning agent is tetracycline or minocycline. The method of tanning the skin according to claim 2 wherein said at least one other tanning agent is sprayed on tanning used after Rifampin tanning method to augments its effects.
